(19) United States
(12) Patent Application Publication
(10) Pub. No.: US 2023/0101601 A1
(43) Pub. Date: Mar. 30, 2023

(54) Method and Device for Evaluating a qPCR Curve

(71) Applicant: Robert Bosch Gesellschaft Mit Beschränkter Haftung, Stuttgart (DE)

(72) Inventors: Christoph Faigle, Muensing (DE); Luay Bannoura, Erlangen (DE)

(21) Appl. No.: 17/904,300

(22) PCT Filed: Feb. 25, 2021
(86) PCT No.: PCT/DE2021/100192

§ 371 Date: Aug. 16, 2022

(30) Foreign Application Priority Data
Feb. 25, 2020 (DE) 10 2020 202 361.1

(51) Int. Cl. C12Q 1/6851; C12Q 1/686; G16B 25/20

(52) U.S. Cl. CPC C12Q 1/6851; C12Q 1/686; G16B 25/20

(57) ABSTRACT

The disclosure relates to a method for carrying out a quantitative polymerase chain reaction (qPCR) process, comprising the following steps:—cyclically carrying out qPCR cycles; —measuring the fluorescence for each qPCR cycle to obtain a qPCR curve of intensity values (I); —creating a probability density function (PDF) from the intensity values (I); —establishing a presence or absence of the DNA strand section to be detected depending on the presence of one or more features of the probability density function (PDF); —carrying out the qPCR process depending on the presence or absence of the DNA strand section to be detected.

### TECHNICAL FIELD

The invention relates to the use of a polymerase chain reaction method (PCR method), especially for detection of the presence of a pathogen. The present invention further relates to the evaluation of qPCR measurements.

### TECHNICAL BACKGROUND

DNA strand segments in a substance to be tested, such as, for example, a serum or the like, are detected by carrying out PCR methods in automated systems. Said PCR systems make it possible to amplify and detect particular DNA strand segments to be detected, for example those which can be assigned to a pathogen. A PCR method generally comprises cyclic use of the steps of denaturation, annealing and elongation. In particular, the PCR process involves splitting of a DNA double strand into individual strands and making each of them complete again by attachment of nucleotides in order to reproduce the DNA strand segments in each cycle.

The qPCR method makes it possible to quantify the pathogen load detected using this process. To this end, at least some of the nucleotides are provided with fluorescent molecules which, upon binding to the individual strand of the DNA strand segment to be detected, activate a fluorescence property. After synthesis of the double strands, an intensity value of a fluorescence that is dependent on the number of DNA strand segments generated can be determined after each cycle.

During amplification, it is then possible to determine from the intensity values determined a qPCR curve which has a sigmoidal shape in the event of the presence of the DNA strand segment to be detected in the substance to be tested. In reality, the qPCR curves measured may contain artifacts, and so multiple parallel measurements are generally carried out in order to make a more accurate evaluation of the qPCR curves possible through averaging of the measurement values.

In particular, it is desirable to identify from a measured qPCR curve whether amplification of DNA strand segments has taken place, and if so, to make an estimation of a starting concentration of the DNA strand segment to be detected.

### DISCLOSURE OF THE INVENTION

According to the invention, a method for carrying out a qPCR method as claimed in claim 1 and a device and a qPCR system as claimed in the alternative independent claims are provided.

Further embodiments are specified in the dependent claims.

According to a first aspect, a method for conducting a quantitative polymerase chain reaction (qPCR) method is provided, comprising the following steps:
cyclic execution of qPCR cycles
measurement of an intensity value after or during each qPCR cycle in order to obtain a qPCR curve composed of intensity values;
creation of a probability density function depending on the intensity values;
establishment of a presence or nonpresence of the DNA strand segment to be detected depending on the presence of one or more features of the probability density function;
conduction of the qPCR method depending on the presence or nonpresence of the DNA strand segment to be detected.

The qPCR method comprises cyclic repetition of the steps of denaturation, annealing and elongation. In the case of denaturation, the entire double-stranded DNA in the substance to be tested is split into two individual strands at a high temperature. In the annealing step, one of the primers added is bound to the individual strands, which primers specify the starting point of amplification of the DNA strand segments to be detected. In the elongation step, a complementary second DNA strand segment is synthesized from free nucleotides on the individual strands provided with the primer. After each of these cycles, the DNA quantity of the DNA strand segments to be detected has thus ideally doubled.

By using the qPCR method, fluorescent molecules are incorporated as labels into the DNA strand segments to be detected, and so it is possible, via measurement of the intensity of the fluorescence after each elongation step, to determine a time plot of the intensity values. The qPCR curve thus obtained comprises three distinct phases, namely a baseline, in which the intensity of the fluorescence of the fluorescent light emitted by incorporated labels is still indistinguishable from the background fluorescence, an exponential phase, in which the fluorescence intensity rises above the baseline, i.e., becomes visible, the doubling of the DNA strands in each cycle causing the fluorescence signal to exponentially rise proportional to the quantity of the DNA strand segments to be detected, and a plateau phase, in which the reagents, i.e., the primer and the free nucleotides, are no longer present in the required concentrations and no further doubling takes place.

For the detection of a specified DNA strand segment to be detected, which can correspond to a pathogen for example, the so-called ct (cycle threshold) value is relevant here. The ct value determines the start of the exponential phase and is determined by exceeding of a specific threshold, which has been defined for whichever DNA strand segment is to be detected and which is identical for all samples for the DNA strand segment to be detected, or is determined mathematically by the second derivative of the qPCR curve in the exponential phase and corresponds to the intensity value of the steepest rise of the qPCR curve. If the target value is known, the starting concentration of the DNA strand segment to be detected in the substance to be tested can be determined by back-calculation.

In reality, the qPCR curves are highly inaccurate and are subject to considerable fluctuations. Baseline drift can occur, which refers to the rise of the background fluorescence above the measurement cycles. This means that, even if no amplification is taking place, the fluorescence signal is rising. Further influencing factors which have an adverse effect on the accuracy of the qPCR curve can, for example, result from thermal noise, fluctuations in the reagent concentration, and air pockets and artifacts in the fluorescence volume.

In conventional qPCR systems, what is done, firstly, is software-based correction of the PCR curves and what can be envisaged, secondly, is repeatedly measuring a sample under the same conditions and smoothing the resultant qPCR curves by averaging. However, this requires increased effort.

It is a concept of the above method to establish, with the aid of a probability density function, the presence of a sigmoidal or linear shape of the qPCR curve. The use of the probability density function can reliably predict, even in the case of qPCR curves greatly affected by noise, whether amplification, i.e., reproduction, of the DNA strand segment to be detected has occurred or whether the qPCR curve is merely rising because of baseline drift.

The probability density function indicates the frequency with which an intensity value occurs. To this end, what is done for the individual data points of the qPCR curve, i.e., for each intensity value of the measured qPCR curve, is to assume a Gaussian distribution around the measured intensity value. Owing to the sigmoidal shape of a qPCR presence curve, i.e., a qPCR curve in which a DNA strand segment has been reproduced, the probability density function has multiple cycles with similar intensity values both in the region of the baseline and in the plateau phase. Therefore, the probability density function of a qPCR presence curve will have two maxima with a minimum between them.

In the case of nonamplification, which yields a qPCR nonpresence curve, i.e., a qPCR curve in which no DNA strand segment has been reproduced, the shape of the qPCR curve is merely determined by the rising of the baseline. This leads to the formation of only one maximum in the probability density function.

In particular, a sigmoidal shape of the qPCR curve can be inferred if the maximum is higher at low intensity values than at high intensity values, a ratio of the minimum between the maxima to the first maximum is below a predetermined threshold and the width of the peak around the second maximum is relatively large.

The above method reliably allows identification of whether amplification has taken place.

If it is identified that the qPCR curve tends to correspond to a sigmoidal shape rather than to a linear shape, a sigmoid function can be fitted to the qPCR curve and the parameters of the sigmoid function can be used to determine a ct value. This allows reliable determination of a ct value even in the case of qPCR curves which are greatly affected by noise and in which amplification has taken place.

Furthermore, the probability density function can be created depending on modified intensity values, wherein the modified intensity values are dependent on or correspond to the intensity values, which have been corrected by a proportion of the fluorescence of a baseline drift of the PCR method.

In particular, the proportion of the fluorescence of the baseline drift can be determined by determining, with the aid of a k-means algorithm, the intensity values to be assigned to a baseline region of the qPCR curve, wherein the intensity values to be assigned to the baseline region are linearized with the aid of linear interpolation and wherein and subsequently the plot of the linearized intensity values of the baseline drift is subtracted from the measured qPCR curve.

In one embodiment, the modified intensity values can be determined by smoothing the measured qPCR curve with the aid of a filter, especially a moving average filter.

It can be envisaged that the presence or nonpresence of the DNA strand segment to be detected is established depending on the presence of at least one of the following features of the probability density function:
the ratio of the function value of the probability density function of the first maximum to the function value of the second maximum is greater than 1;
the ratio of the function value of the local minimum between the maxima to the function value of the first maximum is less than 0.7, especially less than 0.6, and
the width of the peak in the density function around the second maximum is greater than a specified reference value, wherein the width of the peak can be determined as the width of the peak according to the so-called “half-prominence method”. With said method, half of the numerical value of the maximum is first determined. The point which has the same location as the maximum on a horizontal (X) height and has half the numerical value of the maximum on a vertical (Y) height is then referred to as the halfway midpoint. The intersections between the density curve and a horizontal line through the halfway midpoint are then determined. The distance between the two points closest to the halfway midpoint then determines the width of the peak as the reference value.

Furthermore, the qPCR method can be conducted by
signaling that a ct value is determinable and/or
determining the ct value from the parameterized presence function
   if a presence of the DNA strand segment to be detected is established.

### BRIEF DESCRIPTION OF THE DRAWINGS

Embodiments will be more particularly elucidated below on the basis of the accompanying drawings, where:

FIG. 1 shows a schematic depiction of a cycle of a PCR method;

FIG. 2 shows a schematic depiction of a typical qPCR curve comprising a plot of intensity values;

FIG. 3 shows a measured plot of a qPCR curve;

FIGS. 4a and 4b show ideal plots of the qPCR curve in the case of a nondetectable substance and a detectable substance, respectively; and

FIG. 5 shows a flowchart to illustrate a method for conducting a qPCR measurement;

FIG. 6 shows the Gaussian distributions of the individual modified intensity values and the resultant probability density function for a measured qPCR presence curve; and

FIGS. 7a and 7b show, for an ideal qPCR presence curve and an ideal qPCR nonpresence curve, the manifestation of the corresponding probability density function.

### DESCRIPTION OF EMBODIMENTS

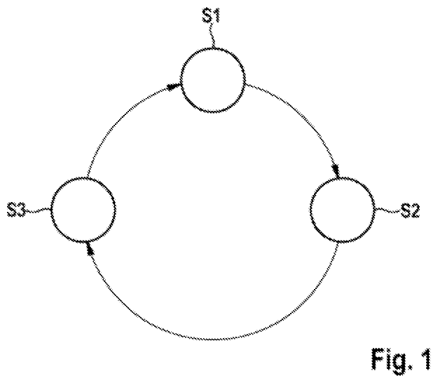

FIG. 1 shows a schematic depiction of a PCR method known per se, comprising the steps of denaturation, annealing and elongation.

In the annealing step S1, the double-stranded DNA in a substance is broken up into two individual strands at a high temperature of, for example, above 90° C. In a subsequent annealing step S2, a so-called primer is bound to the individual strands at a particular DNA position marking the start of a DNA strand segment to be detected. Said primer represents the starting point of an amplification of the DNA strand segment. In an elongation step S3, the complementary DNA strand segment is synthesized on the individual strands from free nucleotides added to the substance, starting at the position marked by the primer, with the result that the previously split individual strands have been completed to form complete double strands at the end of the elongation step.

By providing the free nucleotides or the primer with fluorescent molecules which exhibit fluorescence properties only when bound to the DNA strand segment, it is possible, by determining an intensity of a fluorescence following the elongation step S3, to obtain an intensity value through an appropriate measurement. What is assigned to the measured intensity of the fluorescent light is an intensity value.

The method comprising steps S1 to S3 is executed cyclically and the intensity values are recorded in order to obtain a plot of intensity values as a qPCR curve.

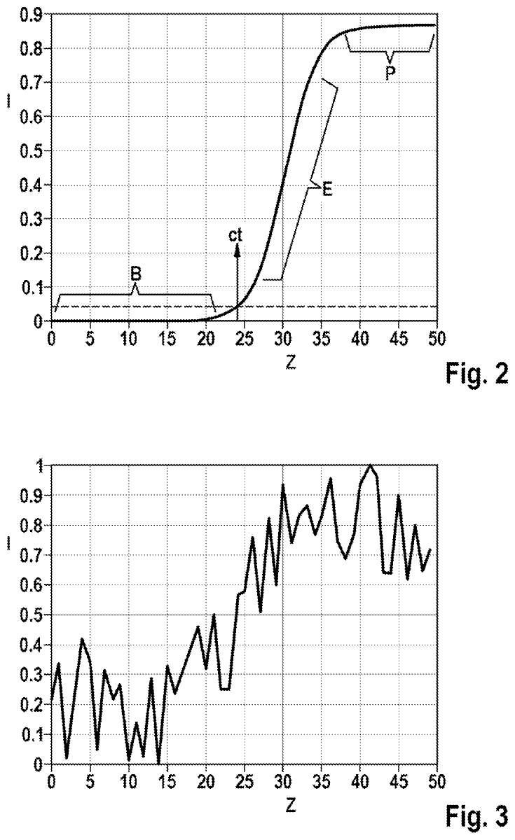

The plot of intensity values ideally has the shape depicted in FIG. 2. FIG. 2 shows a plot of normalized intensity against the cycle index Z. Said plot is divided into three sections, namely a baseline section B, in which the fluorescence of the incorporated fluorescent molecules is still indistinguishable from a background fluorescence, an exponential section E, in which the intensity values are visible and rise exponentially, and in a plateau section P, in which there is flattening of the rise in intensity values, since the reagents used (solution containing nucleotides) have been consumed and no further binding to broken-up individual strands is taking place.

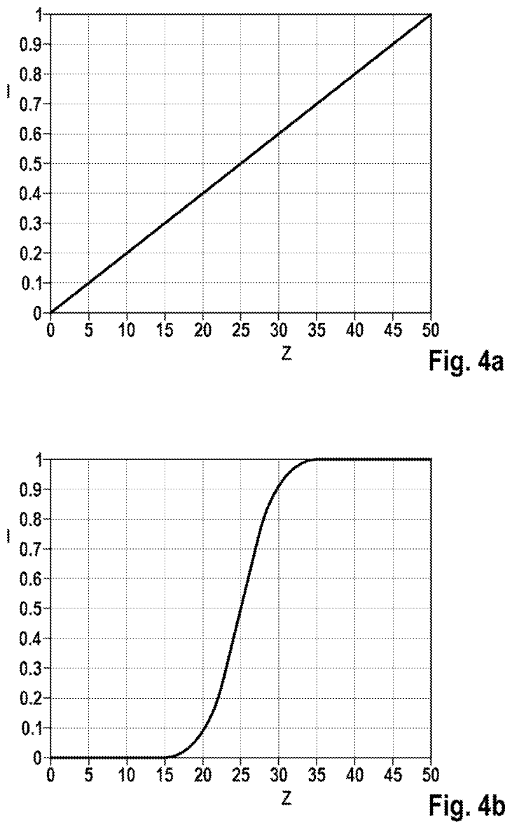

FIG. 3 depicts, by way of example, a plot of the intensity values obtained in a real measurement as a qPCR curve. Strong fluctuations are evident, and these may result from background fluorescence, thermal noise, fluctuations in the reagent concentrations, and bubbles and artifacts in the fluorescence volume. It is evident that it is not readily possible to determine the baseline section, exponential section and plateau section of the qPCR curve.

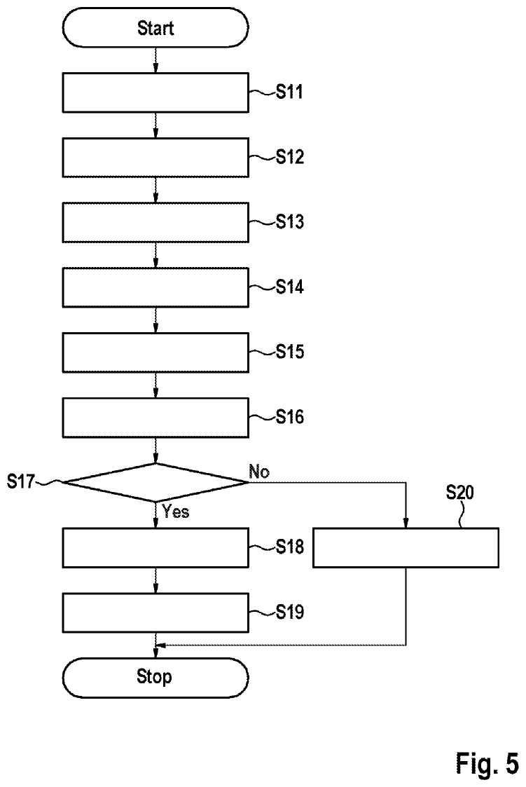

FIGS. 4a and 4b show ideal plots of a qPCR curve without the presence of a DNA strand segment to be detected and with the presence of a DNA strand segment to be detected, respectively.

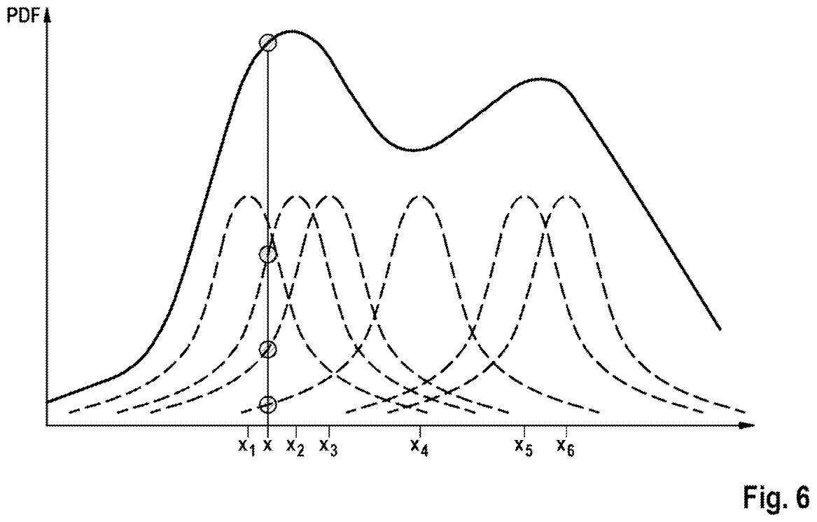

FIG. 5 depicts a flowchart to illustrate a method for evaluating a qPCR curve. The method can be executed on a data processing device which a qPCR process on a qPCR system and which provides from a qPCR system with each cycle an intensity value indicating the intensity of a fluorescence of a substance. In the data processing device, the below-described method can be implemented in software and/or hardware.

In step S11, a qPCR curve comprising the intensity values of a qPCR measurement in a qPCR system is provided. The intensity values are usually measured by capture of a sample using a camera and evaluation of grayscale values/color values and intensity values.

In step S12, the measured qPCR curve is smoothed with the aid of a filter, especially a moving average filter, by assuming for each value the mean of said value and the values of the immediate intensity values recorded subsequently, such as, for example, the preceding and subsequent two to five neighboring values.

In step S13, a clustering algorithm is used to determine three curve regions: baseline region, exponential region and plateau-phase region. To this end, a baseline centroid, an exponential-region centroid and a plateau-region centroid are initially placed in the graph of the qPCR curve. The initial centroids can be positioned in the approximate positions thereof owing to knowledge of the shape of the sigmoid function. Using prior knowledge of the baseline region being located at low intensity values, the exponential region being located at medium intensity values and the plateau region being located at high intensity values, it is possible to place the baseline centroid C1, the exponential-region centroid C2 and the plateau-region centroid C3. Thereafter, every point on the measured qPCR curve is assigned to the nearest centroid and classified thereby.

The k-means algorithm provides an iterative adjustment of the centroid points C by forming a mean of the measurement points of the qPCR curve that have been assigned to a respective centroid.

$${C}_{k} = \frac{1}{\vert {S}_{k} \vert} {\sum }_{x ∈ {S}_{k}} {x}^{( i )} with k = 1 , 2 , 3 , …$$

(S_{K}: number of measurement points assigned to the respective centroid) The measurement points of the qPCR curve can now be reassigned to the altered centroid points with the aid of distance determination

Dⱼ=\sqrt{(x⁽ⁱ⁾−cⱼ)²} for j=1, . . . ,k

and with

A⁽ⁱ⁾=j for Dⱼ minimum

This method is executed iteratively until there is no more change in the assignment of points to clusters or until a maximum number of iterations has been reached.

Subsequently, every measurement point of the measured qPCR curve is reassigned to the respectively redetermined centroid point of the baseline centroid, the exponential-region centroid and the plateau-region centroid.

In step S14, the points of the qPCR curve that have been assigned to the baseline region, i.e., to the determined baseline centroid point, can be used to create a linear curve of intensity values by interpolation. The plot of the linearized qPCR baseline curve corresponds to the influence of the baseline plot on the entire qPCR measurement. Therefore, the linearized qPCR baseline curve is subtracted from the entire measured qPCR curve. This eliminates the baseline rise from the qPCR curve.

In the next step S15, the remaining qPCR curve is normalized, so that the points of the qPCR curve lie as modified intensity values between 0 and 1.

Thereafter, in step S16, a probability density function is created. It indicates the probabilities of the occurrence of modified intensity values in the normalized linearized qPCR curve. To this end, the modified intensity value for each cycle is provided with a Gaussian distribution around the corresponding modified intensity value. The probability density function corresponds to the sum of all Gaussian distributions of the modified intensity values.

In the case of a successful amplification, multiple cycles having similar modified intensity values are present both in the baseline region and in the plateau region. When the Gaussian distributions relating to the probability density function are summated, this leads to two characteristic maxima. By contrast, in the case of a nonamplification, only the baseline determines the plot of the qPCR curve, and so a manifestation of two maxima is essentially not to be expected.

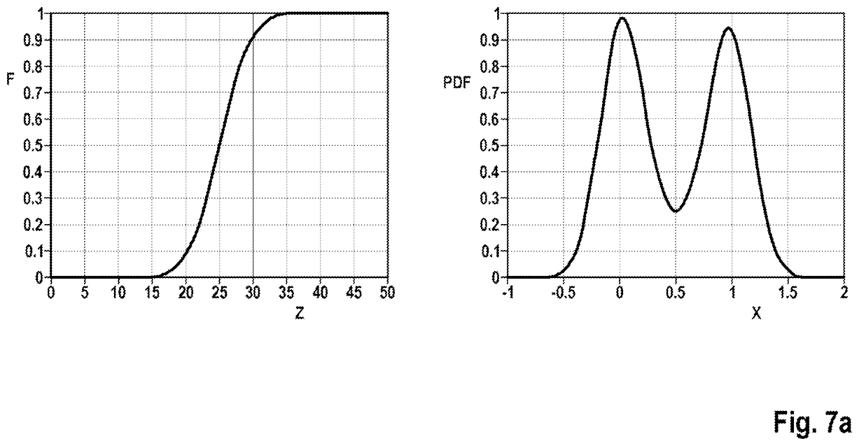

The graph of FIG. 6 shows, by way of example, the Gaussian distributions of the individual modified intensity values x and the resultant probability density function PDF for a measured qPCR presence curve.

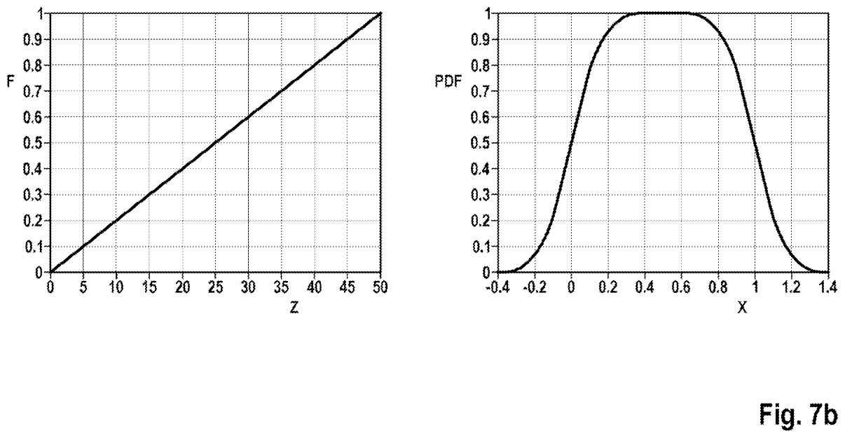

FIGS. 7a and 7b show, for an ideal qPCR presence curve and an ideal qPCR nonpresence curve (left-hand curve in both cases, with the modified intensity value F plotted against the cycle index z), the manifestation of the corresponding probability density function (right-hand curve).

Owing to noise in the nonamplified case, two maxima of the probability density function may likewise occur. Nevertheless, it is possible to distinguish between the event of amplification and the event of nonamplification if at least one of the following criteria is present:
the maximum of the probability density function for low intensity values is higher than the maximum of the probability density function for higher intensity values;
there is a pronounced local minimum between the two maxima;
the width of the second maximum is relatively high.

In step S17, a check is made as to whether the resultant probability density function has its origin in a qPCR presence curve. This can be carried out by checking whether the ratio of the height (function value of the probability density function) of the first maximum to the height of the second maximum is greater than 1, the ratio of the height of the local minimum between the maxima to the height of the first maximum is less than 0.7, especially less than 0.6, and that the width of the peak around the second maximum is greater than a reference value, such as 8 for example.

The width of the reference value is obtained using the so-called “width half prominence” method for a probability density distribution plotted on a normalized scale from 0 to 100. With said method, half of the numerical value of the maximum is first determined. The point which has the same location as the maximum on a horizontal (X) height and has half the numerical value of the maximum on a vertical (Y) height is then referred to as the halfway midpoint. The intersections between the probability density function and a horizontal line through the halfway midpoint are then determined. The distance between the two points closest to the halfway midpoint then determines the width of the peak. The reference value can differ depending on the use of different probability density distributions and methods for plotting of the density.

If it is established in step S17 that the resultant probability density function has its origin in a qPCR presence curve (alternative: yes), a sigmoid function can be fitted to the qPCR curve in step S18, according to the following rule:

$$Y = \frac{{F}_{m a x}}{1 + {e}^{- \frac{x - {x}_{0.5}}{k}}} + {F}_{b}$$

In the next step S19, the ct value can then be determined in a manner known per se through the maximum of the second derivative of the fitted sigmoid function.

If it is established in step S17 that the resultant probability density function has its origins in a qPCR presence curve (alternative: no), a nonpresence of the strand segment to be detected can be signaled in step S20.

1. A method for conducting a quantitative polymerase chain reaction (qPCR) process, the method comprising:
cyclically executing of qPCR cycles;
measuring a fluorescence at each qPCR cycle to obtain a qPCR curve composed of intensity values;
creating a probability density function from the intensity values of the qPCR curve;
establishing one of a presence and a nonpresence of a DNA strand segment to be detected depending on a presence of at least one feature of the probability density function;
conducting the qPCR process depending on the one of the presence and the nonpresence of the DNA strand segment to be detected.

2. The method as claimed in claim 1, the creating further comprising:
creating the probability density function depending on modified intensity values, the modified intensity values being one of (i) dependent on the intensity values and (ii) corresponding to the intensity values, the modified intensity values having been corrected by a proportion of a fluorescence of a baseline drift curve of the qPCR process.

3. The method as claimed in claim 2, further comprising:
determining the proportion of the fluorescence of the baseline drift by determining, with a clustering algorithm, intensity values to be assigned to a baseline region of the qPCR curve,
linearizing the intensity values to be assigned to the baseline region with linear interpolation and
subtracting, subsequently a plot of the linearized intensity values of the baseline drift from the qPCR curve.

4. The method as claimed in claim 2, wherein further comprising:
determining the modified intensity values by smoothing the qPCR curve with a filter.

5. The method as claimed in claim 1, wherein the one of the presence and the nonpresence of the DNA strand segment to be detected is established depending on a presence of at least one of the following features of the probability density function:
a ratio of a function value of the probability density function of a first maximum to a function value of a second maximum is greater than 1;
a ratio of a function value of a local minimum between a maxima to a function value of the first maximum is less than 0.7; and
a width of a peak in the probability density function around the second maximum is greater than a specified reference value.

6. The method as claimed in claim 1, the conducting the qPCR process further comprising at least one of:
signaling that a ct value is determinable; and
determining the ct value from a parameterized presence function in response to the presence of the DNA strand segment to be detected being established.

7. A device for conducting a quantitative polymerase chain reaction (qPCR) process, the device being configured to:
cyclically execute qPCR cycles;
measure a fluorescence at each qPCR cycle to obtain a qPCR curve composed of intensity values;
create a probability density function from the intensity values of the qPCR curve;
establish one of a presence and a nonpresence of a DNA strand segment to be detected depending on a presence of at least one feature of the probability density function; and
conduct the qPCR process depending on the one of the presence and the nonpresence of the DNA strand segment to be detected.

8. The method as claimed in claim 1, wherein the method is carried out by executing a computer program.

9. A non-transitory electronic storage medium storing a computer program for conducting a quantitative polymerase chain reaction (qPCR) process, the computer program being configured to, when executed by a computer, cause the computer to:
cyclically execute qPCR cycles;
measure a fluorescence at each qPCR cycle to obtain a qPCR curve composed of intensity values;
create a probability density function from the intensity values of the qPCR curve;
establish one of a presence and a nonpresence of a DNA strand segment to be detected depending on a presence of at least one of the probability density function; and
conduct the qPCR process depending on the one of the presence and the nonpresence of the DNA strand segment to be detected.

10. The method as claimed in claim 4, wherein the filter is a moving average filter.

11. The method as claimed in claim 5, wherein the one of the presence and the nonpresence of the DNA strand segment to be detected is established depending on a presence of the following feature of the probability density function:
the ratio of the function value of the local minimum between the maxima to the function value of the first maximum is less than 0.6.
